(19) United States
(12) Patent Application Publication
(10) Pub. No.: US 2026/0248595 A1
(43) Pub. Date: Aug. 27, 2026

(54) NON-OSTEOCONDUCTIVE INORGANIC DEVICE FOR BONE FORMATION, BONE RESHAPING OR HEALING OF BONE

(71) Applicant: Medcer AB, Källered (SE)

(72) Inventor: Johan MALMSTRÖM, Varberg (SE)

(21) Appl. No.: 19/159,404

(22) PCT Filed: Feb. 28, 2024
(86) PCT No.: PCT/SE2024/050186

§ 371 Date: Aug. 25, 2025

(30) Foreign Application Priority Data
Mar. 3, 2023 (SE) 2330110-4

(51) Int. Cl. A61C 8/02; A61C 1/08; A61C 8/00

(52) U.S. Cl. CPC A61C 8/0006; A61C 1/084; A61C 8/0012; A61C 8/008

(57) ABSTRACT

The invention relates to a device for bone formation, bone reshaping or healing of bone in dental, oral and maxillofacial applications, specifically in connection with installation of dental implants. The device comprises a sheet structure made of an inert material, wherein the sheet structure has an inner surface intended to be directed towards the bone and an outer surface intended to be directed away from the bone. The geometrical features of the cavity formed between the inner surface of the device and the initial bone surface when the device is installed at the intended location is further used to improve and plan the treatment. The device is customized by means of digital planning based on the initial and the optimal bone volume for the intended treatment. Modifications of the device can be introduced to facilitate a combined installation of dental implants with the device.

### CROSS-REFERENCE TO RELATED APPLICATIONS

This Application is a Section 371 National Stage Application of International Application No. PCT/SE2024/050186, filed Feb. 28, 2024 and published as WO/2024/186247 on Sep. 12, 2024, in English which claims priority to SE 2330110-4, filed Mar. 3, 2023, the contents of which are hereby incorporated by reference in their entirety.

### FIELD OF THE INVENTION

The present invention relates to a device for bone formation, bone reshaping or healing of bone in oral and maxillofacial applications, specifically in connection with the installation of dental implants.

### BACKGROUND OF THE INVENTION

Tooth loss can occur due to various reasons such as poor dental care, other health issues or trauma. A couple of solutions to replace missing teeth have been developed, since the demand of functional teeth at all stages of life is growing. Dental implants are one such option that can be used to replace missing teeth in various situations, from a single tooth to the entire set of teeth. Titanium screws are often used as dental implants, placed within the jawbone allowing a crown, bridge or denture to be attached to it. This is a rapidly evolving field where new technologies and materials have enabled the development of implant systems that ensure faster healing, more reliable results and improved esthetical outcome. However, the prognosis for implant survival depends also on the bone volume and bone quality surrounding the implant. This means that if the bone volume (width, depth, height) is insufficient or the anatomical conditions are unsuitable for an implant installation, the situation need to be changed and there are several different solutions that can be used to increase the bone volume.

A common solution to enhance the bone volume is to use particulate bone graft materials. These materials can be of either natural or synthetic origin with a chemical composition similar to the inorganic part of the bone tissue and consists often of calcium phosphate based compounds. The bone cells can attach to these materials and grow along the surface (osteoconductive materials). The intention is to form new bone on the particle surface as well as in the space between the particles. However, the initial shape of the structure formed by loose particles may change when chewing or during other types of motions and result in that the desired geometrical shape of the bone volume to be regenerated is not always predictable. For larger bone volumes, graft particles are often combined with a membrane with the purpose to enclose and maintain the shape of the graft material. These membranes are often made of soft flexible organic materials such as collagen or Gortex or thin metallic sheets that can be cut and adjusted during surgery to fit the patient. The organic membrane materials are too soft to be able to protect the enclosed volume from external forces that is transferred and cause motions in the graft material and result in a less favorable situation for the growing bone. Even though the organic membrane materials used are biocompatible, they often release molecules that contribute to some soft tissue reaction. The metallic membranes are often made of perforated titanium sheets to allow the sheet to be bent and customised. Titanium has been found to work excellent in combination with bone tissue and bone anchored implants since the material has osseoconductive properties. However, the thin Titanium sheets are also in contact with the soft tissue, where sharp edges are less favorable and may cause soft tissue penetration. There are thus a couple of limitations of the systems used today related to customisation, clinical function of the membranes and the ability to maintain the shape of the volume where new bone is intended to be formed. It would thus be desired to have a solution that give a predictable result and improves the esthetical outcome of the treatment.

There are rather few customised solutions for bone regeneration that have been presented and most of these are made with additive manufacturing of titanium mesh structures. These manufacturing techniques gives usually a relatively rough surface with Ra values that has been reported to vary between 0.6 to 6 μm. The osseoconductive properties of the titanium mesh structures result further in that the bone can attach and grow into the structure, which generates additional work during the removal of the temporary membrane.

### SUMMARY

It is an object of the present invention to provide a customised device of an inert material that contributes to an enhanced bone formation with a predictable geometrical shape when the bone volume is increased, reshaped or healed, preferably in dental, oral and maxillofacial applications. The invention can also be applied to other types of bone reconstructions where the loading capacity, anatomical shape or the function of the skeleton needs to be restored. The device can be used as a pre-treatment before dental implants are installed or in combination with the installation of the implants where the design of the device is defined by a combination of the original bone structure and the desired bone structure to be formed. The customisation of the device will also include the location and design of the fixation points where fixation screws can be used to fasten the device to the bone as well as modifications of the device to facilitate a combined installation of implants together with the device.

According to the invention the initial shape of the bone structure can be obtained from computer tomography or similar techniques to generate a three dimensional model of the bone structure. Additional bone can then be added to the digital model until the desired bone volume for the intended treatment has been reached. The desired bone volume will then be formed by the initial bone volume and the bone volume to be regenerated. This means that when the customised device is installed at the intended location, the edges of the device will follow and be in close contact with the initial bone structure. A cavity is formed between the inner surface of the device and the initial bone structure that corresponds to the bone volume to be regenerated. For practical reasons, the entire edge of the device may not follow the initial bone structure.

The customised device can further be used in an augmentation procedure before dental implants are installed, in combination with the installation of dental implants or to modify the geometrical shape of the bone or to generate bone for other reasons. The shape and volume of the bone to be formed can be designed through the inner surface of the device directed towards the bone. To enhance the bone formation, bone grafting material can be added to the cavity. The volume of the cavity can be obtained from the digital model and used to choose a suitable amount of bone graft material to be used. Implants can be installed in connection with the device even if they are not fully stabilized by the initial bone volume present. If an implant is installed inside the device, the height of the cavity at the location of the implant will define the installation depth in the initial bone structure. The device is preferably fixated to the bone by small screws located at anatomical suitable locations, preferably identified from the model of the bone structure. Holes can also be added to the device that can be used as a backup option for additional fixation screws to ensure that a stable fixation can be obtained or for inspection of the bone level reached during healing before the device is removed or to allow transfer of substances, liquids and materials to the cavity after installation.

### BRIEF DESCRIPTION OF THE DRAWINGS

In the following the invention will be described more in detail with reference to the accompanying drawings in which,

FIG. 1 shows a cross section from the side through the initial bone structure, the desired bone structure and the customized device, where an implant was installed inside the device to a depth in the initial bone defined by an average distance from the original bone to the new bone level. The customised device is further fixated to the original bone by fixation screws.

FIG. 2 shows a cross section through the device around the top region of a dental implant with different design options that can be used to facilitate a dental implant to be installed together with the device.

FIG. 3 shows the front view of an oral situation where the implant planning is done to define the desired bone level and the inner surface of the customised device. The implants can be installed with the device, or when the device is removed in the new bone formed.

FIG. 4 shows an example of a customised device, designed from patient specific digital data.

### DETAILED DESCRIPTION OF EXAMPLE EMBODIMENTS

A specific embodiment of the invention will now be described with reference to the accompanying drawings. This invention may, however, be embodied in many different forms and should not be construed as limited to the embodiment set forth herein.

The invention relates to a customised device consisting of an inorganic inert material used to regenerate bone tissue with a digitally designed shape and volume, preferably in combination with a bone graft material. The customisation of the device is based on digital data of the initial bone structure and an additional bone volume to support the intended treatment with respect to the esthetical outcome as well as the mechanical requirements. This means that the bone structure can be designed with respect to height, width and slopes to give an optimal mechanical and esthetical solution based on the preferred implant location and angulation.

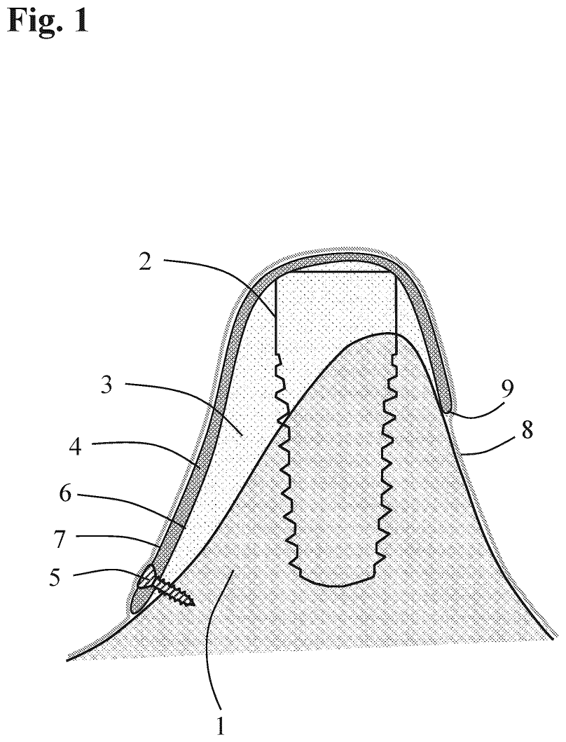

According to the invention, the customised device as shown in FIG. 1-4 has an inner surface directed towards the bone that will correspond to the surface of the desired bone structure and an outer surface directed towards the soft tissue. The inner and outer surface of the device is connected by an external boundary that follows the surface of the initial bone structure. When the device is installed, the external boundary of the device will be in contact with the initial bone structure and is further used to define the location when fixated to the bone. Due to practical reasons, there can be a gap between the external boundary of the device and the bone structure along some parts of the external boundary of the device.

The geometrical features such as the cavity volume, height at the intended location of the implant, the shape of the cavity formed between the inner surface of the device and the initial bone can be used to improve the clinical result. When the clinical use of the device is combined with a bone graft material, the volume of bone graft used would preferably be comparable to the volume of the cavity or slightly less. A too small amount would result in a partly filled volume while a too large volume would result in a difficulty to find the correct position when the device is installed. A known cavity volume can then facilitate the procedure to choose a suitable amount of bone graft material to be used. The cavity height can further be used to define the drilling and the implant installation depth in the initial bone if the implants are installed together with the device.

When the shape of the device has been defined and customised to the desired bone structure, there are also some other aspects that can contribute to additional design modifications of the device. The size, location and shape of the holes for fixation of the device to the bone or for other purposes such as inspection of the bone level, transfer of substances to the cavity formed is preferably defined in the digital planning where anatomical or for other reasons unsuitable locations are avoided. If implants are planned to be installed during the same procedure as the device, additional geometrical modifications can be made to facilitate the implant installation. The inner surface of the device can be raised in the region surrounding the upper part of the implant to be installed to ensure a margin to avoid a direct contact between the device and the implant, to allow space for a healing cap attached to the implant and to allow the correct position with respect to the bone level to be reached. This can be achieved either by making the device thinner or through an increased height of the device. In both cases the inner surface around the upper part of an installed implant will deviate from the intended bone surface. There is further an option to make a small hole in the device to allow the implant height to be adjusted or a larger hole for the implant or the healing cap attached to the implant to be extended above the bone level.

When the device is installed between the soft tissue and the initial bone structure, the soft tissue has to be cut and opened to expose the bone. The device can then be installed at the correct location with or without additional bone graft materials and fixated before the soft tissue is returned and sutured to enclose the device. Without osseoconductive properties, the bone growth will not be promoted to follow the inner surface of the device, bone growth will instead occur from the surface of the initial bone structure towards the inner surface of the device where a thin periost is formed between the bone and the device. The absence of osseoconductive properties result further in that the new bone formed will not bond to the inner surface of the device and facilitate the removal procedure of the device. When large bone volumes are to be regenerated, additional bone graft materials can preferably be added in the cavity formed between the device and the initial bone to support the bone formation. When the device is removed and there are no surrounding teeth, the shape of new bone structure, can be used to support the installation of dental implants with the correct location and angulation using a drill guide with a shape that correspond to the shape of the inner surface of the device and the initial bone.

The device can be composed of various materials without osteoconductive properties such as silicon nitride, alumina, titania, zirconia, aluminates or composites based on these materials. There are several fabrication methods that can be used to generate the desired macroscopic shape of the device such as machining from a blank (green, presintered or sintered), various casting methods such as slip casting, gel casting, additive manufacturing, injection moulding. The size of the device and all features of the device are adjusted to compensate for dimensional changes during the manufacturing process of the customised device to ensure a perfect match to the patient. All edges of the device are preferably chamfered or rounded in the manufacturing process.

FIG. 1 illustrates a cross section through the initial bone structure 1 where the bone volume is not sufficient for a stable installation of a dental implant 2. An increased bone level 3 would then be desired to facilitate the installation of the implant and to improve the esthetical result. A device 4 according to the invention for reshaping, healing or regeneration of bone can then be designed based on the initial and the desired bone level 3, defined by the treatment planed. The device comprises a customised structure 4 as shown in the figure that is attached to the bone by fixation screw 5. The device has an inner side/surface 6 intended to be directed towards the bone 1 and an outer side/surface 7 intended to be in contact with the soft tissue 8, an edge 9 connects the inner and outer surfaces 6, 7 of the device and forms the boundary where the soft tissue is separated from the bone tissue. New bone will be formed in the cavity 3 and the outer shape of the new bone can thus be defined by the shape of the inner surface 6 of the device 4. To further enhance the bone regeneration, a bone graft material can be introduced in the cavity. The amount of bone graft material used is preferably comparable too or slightly less than the volume of the cavity formed. It is further possible to install implants together with the device even if the initial bone volume is not sufficient for a stable installation since the device will protect the implant during the time for new bone to be formed and contribute to the stability of the implant. Such a procedure can reduce both the time and the number of surgical procedures needed for the treatment. When the shape of the device has been defined for a certain treatment, it may allow implants to be installed together with the device. The installation depth can be defined by the height of the cavity at the location for the implant to be installed and to avoid a direct contact between the implant and the inner surface of the device.

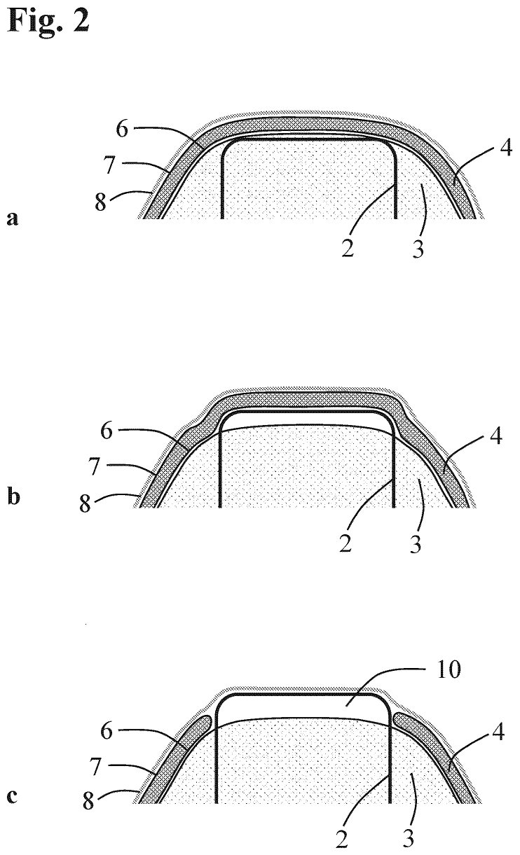

In FIG. 2, some options are illustrated to allow an increased flexibility of the installation height in relation to the desired bone level. This can be achieved by raising the inner surface in the region of the upper part of the dental implant, either a reduction of the thickness of the device or a subtraction of material at the inner side and an addition of material at the outer side to ensure the mechanical reliability of the device. In these cases, the inner surface of the device will not correspond to the desired bone level. This can further be used to increase the distance between the installed implant and the device and allow small geometrical deviations during the installation of the implant and still ensure that the device can be placed at the correct location without being in contact with the implant. It is further possible to make a hole 10 in the device to allow the implant and a healing cap to penetrate the device or to install the device before the implants are installed. With such a design the edges surrounding the holes need to be rounded.

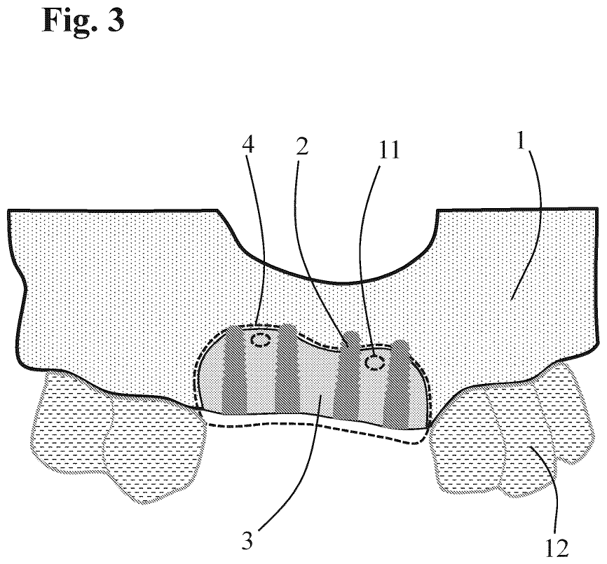

FIG. 3 shows a front view of the maxilla with the initial bone 1, the surrounding teeth 12 and the location and orientation of the implants 2, which are used to define the desired bone volume 3 corresponding to the inner surface of the device 4. Holes 11 for the fixation screws can be located at suitable positions in relation to the device, the bone, anatomical or other practical aspects.

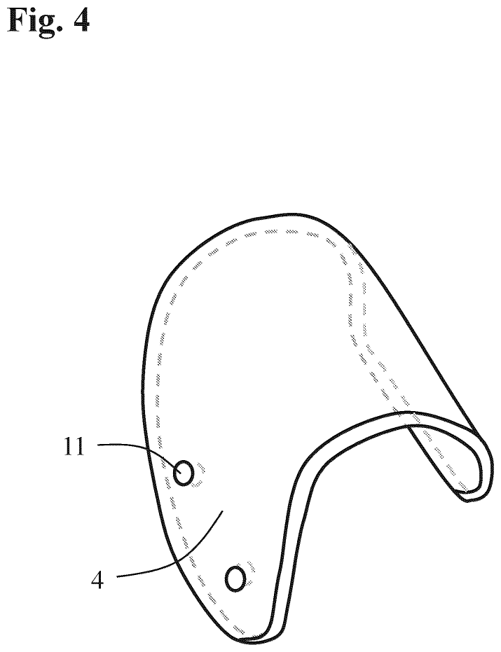

The digital planned implant treatment as illustrated in FIG. 1-3 can be used to define the new bone (height, width and curvatures) for an optimized esthetical outcome and thus also the inner shape of the customised device 4 as shown in FIG. 4. The device 4 can be fixated to the bone structure by screws 5 or fixation pins, inserted through small holes 11 in the device. The size and shape of additional holes, not shown in the figures will depend on the intended use of the hole, which can be for fixation screws, bone level control or material transfer to or from the cavity. The holes are preferably countersunk from the outer surface 7 to reduce the exposure of the screw head and sharp edges towards the soft tissue. As a complement or alternative to the screws and fixation means, the device 4 can be fixed by means of glue.

1. A customised device for installation between soft tissue and an initial bone structure to contribute to enhanced bone formation, reshaping of bone, or healing of bone, the device being made of an inert inorganic material without osseoconductive properties, and having an inner surface arranged to be directed towards the initial bone structure when the device has been installed, the inner surface being shaped to define an outer shape of new bone, and an outer surface arranged to be directed towards the soft tissue when the device has been installed.

2. The device according to claim 1 wherein the inner surface is raised in a region to accommodate an upper part of a dental implant to be installed together with the device.

3. The device according to claim 1, comprising a hole through the device at an intended location of a dental implant.

4. The device according to claim 1, wherein an edge connects the inner and outer surfaces, which edge forms a boundary where the soft tissue is separated from the bone tissue when the device has been installed.

5. The device according to claim 1, wherein the device is composed of an inorganic material such as silicon nitride, alumina, titania, zirconia, aluminates or composites based on these materials.

6. The device according to claim 1 wherein the inner surface defines a shape of a drill guide for installation of dental implants to be placed on the new bone structure.
